(19) United States
(12) Patent Application Publication
(10) Pub. No.: US 2010/0112021 A1
(43) Pub. Date: May 6, 2010

(54) SUSTAINED-RELEASE COMPOSITION

(76) Inventors: Xiaodong Zhang, Livingston, NJ (US); Michael Meerbote, Gutenberg (DE); Ashish Batra, Lake Jackson, TX (US); Linda A. Kosensky, Manville, NJ (US); Klaus Schiller, Halle (DE); Elke Nieter, Hollen (DE)

Correspondence Address: The Dow Chemical Company, Intellectual Property Section, P.O. Box 1967, Midland, MI, 48641-1967, US

(21) Appl. No.: 12/532,218

(22) PCT Filed: Mar. 19, 2008
(86) PCT No.: PCT/US08/57444

§ 371 Date: Sep. 21, 2009

Related U.S. Application Data
(60) Provisional application No. 60/919,222, filed on Mar. 21, 2007.

(51) Int. Cl. A61K 8/81; A61K 47/32; A61K 9/14; A61P 17/00; A61Q 5/00; A61Q 19/00

(52) U.S. Cl. 424/401; 424/70.16; 514/772.6; 424/501

(57) ABSTRACT

A composition which comprises (A) a plurality of cross-linked polymer particles, said polymer being the polymerization product of at least two monomer units selected from the group consisting of monoalkenyl aromatic compounds, alkyl esters derived from a saturated alcohol and acrylic or methacrylic acid, and vinyl esters of an aliphatic carboxylic acid; and said cross-line polymer particles being loaded with (B) an active ingredient, the weight ratio of the active ingredient (B) to the polymer particles (A) being from 0.05 to 50:1, is useful to release the active ingredient over an extended period of time while controlling sebum on skin and hair.

### FIELD OF THE INVENTION

The present invention relates to a composition which allows the sustained release of an active ingredient from the composition.

### BACKGROUND OF THE INVENTION

The sustained release of an active agent, such as a drug, improves the safety, efficacy and reliability of a treatment regimen that utilizes the active agent. Accordingly, sustained-release compositions are widely used in the pharmaceutical field. Sustained-release compositions so far are less widely known in the personal care industry, but it would be highly desirably to also provide personal care compositions which allow sustained release of an active ingredient, such vitamins, fragrances, emollients and sunscreens.

U.S. Pat. No. 6,491,953 discusses the difficulty of achieving a controlled release of an oil-soluble active agent when the oil-soluble active agent is a component of an oil-based controlled release composition or when the oil-soluble active agent in its controlled release form is subjected to a nonaqueous medium. U.S. Pat. No. 6,491,953 tries to overcome the problem of premature release of an active agent from controlled release compositions by providing a composition comprising (a) microparticles from an adsorbent polymer which are free of a monounsaturated monomer and which have a mean unit particle size of 5-80 microns and a bulk density of 0.008-0.1 g/cc and wherein the microparticles are in the form of open spheres and sections of spheres; (b) an oil-soluble topically active compound adsorbed onto said adsorbent polymer microparticles; and (c) a water-soluble release retardant which is coated and adsorbed onto the adsorbent polymer microparticles and the active compound. Delayed release of salicylic acid in Polypore E (a copolymer of allyl methacrylate and ethylene glycol dimethacrylate) coated with stearyl alcohol as a water-soluble release retardant is shown. However, the use of this water-soluble release retardant or another monomeric release retardant as disclosed in U.S. Pat. No. 6,491,953 is not desirable in many controlled release compositions.

Accordingly, it would be desirable to provide new sustained-release compositions which are not dependent on the inclusion of a monomeric release retardant.

### SUMMARY OF THE INVENTION

One aspect of the present invention is a sustained-release composition which comprises

(A) a plurality of cross-linked polymer particles, said polymer being the polymerization product of at least two monomer units selected from the group consisting of monoalkenyl aromatic compounds, alkyl esters derived from a saturated alcohol and acrylic or methacrylic acid, and vinyl esters of an aliphatic carboxylic acid; and said cross-linked polymer particles being loaded with

(B) an active ingredient, the weight ratio of the active ingredient (B) to the polymer particles (A) being from 0.05-50:1.

Another aspect of the present invention is a method of releasing an active ingredient to skin or hair over a time period which method comprises contacting the skin or hair with the above-disclosed sustained-release composition.

Yet another aspect of the present invention is a plurality of cross-linked polymer particles (A), wherein said polymer is the polymerization product of at least two monomer units selected from the group consisting of monoalkenyl aromatic compounds, alkyl esters derived from a saturated alcohol and acrylic or methacrylic acid, and vinyl esters of an aliphatic carboxylic acid; said cross-linked polymer particles (A) are loaded with an active ingredient (B), and the weight ratio of the active ingredient (B) to the polymer particles (A) is from 0.05 to 50:1.

### SHORT DESCRIPTION OF THE DRAWINGS

FIGS. 1 and 2 illustrate the loading of various cross-linked polymer particles with various active ingredients.

FIGS. 3-5 illustrate the release of various active ingredients from various cross-linked polymer particles over time.

FIGS. 6 and 7 illustrate the release of an active ingredient from cross-linked polymer particles comprised in a lotion and its penetration across a silicone membrane that mimics human skin in comparison with the release of an active ingredient from a control lotion that does not comprise cross-linked polymer particles.

FIG. 8 illustrates the sebum control of a sustained-release composition of the present invention and of a control composition for various amounts of sebum applied.

### DETAILED DESCRIPTION OF THE INVENTION

It has surprisingly been found that the composition of the present invention which comprises (A) a plurality of cross-linked polymer particles described further below which are loaded with (B) an active ingredient, the weight ratio of the active ingredient (B) to the polymer particles (A) being from 0.05 to 50:1, is capable of releasing the active ingredient over a period of time allowing for a sustained effect. Accordingly, the composition of the present invention is designated as sustained-release composition. In a preferred aspect of the present invention the composition is a skin care or hair care composition which releases the active ingredient over a period of time when applied to skin or hair Even more surprisingly, it has been found that the preferred skin care or hair care compositions are useful for imbibing sebum thus suppressing oily shine on skin and hair while simultaneously delivering an active ingredient to the hair or skin over an extended period of time.

The cross-linked polymer particles and methods of preparing them are described in U.S. Pat. Nos. 4,489,058 and 4,619,826. These patents disclose the use of the cross-linked polymers for controlling acne. The polymers are able to imbibe and retain sebum. The International Publications WO 92/00719 and WO 92/00724 disclose a makeup composition and a cosmetic lotion comprising the above-mentioned cross-linked polymer. However, none of these prior art documents discloses the benefit of loading an active ingredient on the polymer particles at a weight ratio of the active ingredient (B) to the polymer (A) from 0.05 to 50:1. None of these prior art documents discloses a sustained-release composition that releases the active ingredient over a period of time. To the contrary, WO 92/00719 and WO 92/00724 teach not to include materials of a certain solubility parameter in the composition.

The sustained-release composition comprises a plurality of cross-linked polymer particles (A) which are loaded with an active ingredient (B), wherein the weight ratio of the active ingredient (B) to the polymer particles (A) is from 0.05 to 50:1, preferably from 0.2 to 20:1, more preferably from 0.5 to 10:1, most preferably from 1 to 5:1. The weight ratio above is defined as the total weight of one or more active ingredients (B) divided by the total weight of the cross-linked polymer particles (A) in their un-loaded state.

The cross-linked polymer (A) is the polymerization product of at least two monomer units selected from the group consisting of monoalkenyl aromatic compounds, alkyl esters derived from a saturated alcohol and acrylic or methacrylic acid, and vinyl esters of an aliphatic carboxylic acid. Preferably the cross-linked polymer (A) is the polymerization product of two of the above-mentioned monomer units, the amount of each of the monomer units being from 25 to 75 weight percent, more preferably from 30 to 70 weight percent, based on the total weight of the monomer units. In addition to these monomer units the cross-linked polymer (A) generally comprises a minor amount of the cross-linking agent as described further below. The cross-linking agent can be any di- or poly-functional compound. The cross-linked polymer (A) does not comprise a monomer unit with more than one polymerizable double bond other than the cross-linking agent.

The preferred monoalkenyl aromatic compounds which may be utilized in the preparation of the polymers for the sustained-release composition of the present invention contain a straight or branched chain monoalkenyl residue of from 2 to about 10 carbon atoms and may optionally be ring substituted with halogen or a straight or branched chain alkyl moiety of from 1 to about 20 carbon atoms, more preferably from 1 to about 12 carbon atoms. Such compounds include, for example, various halostyrenes such as 2-chlorostyrene, 3-fluorostyrene, 4-fluorostyrene and the like; vinyl naphthalenes, allylbenzene, 2-phenyl-2-butene, styrene and various substituted styrenes such as alkylstyrenes. Such alkylstyrenes include, for example, n-alkylstyrenes such as methylstyrene (i.e., vinyl toluene), n-butylstyrene, n-amylstyrene, n-octylstyrene, or n-octadecylstyrene; isoalkylstyrenes such as isobutylstyrene, isohexylstyrene, or isododecylstyrene; sec-alkylstyrenes such as sec-butylstyrene, sec-hexylstyrene, or sec-octylstyrene; tertiary-alkylstyrenes such as tert-butylstyrene, tert-amylstyrene, 3,5-ditert-butylstyrene, 4-tert-hexylstyrene, tert-octylstyrene, or tert-eicosylstyrene. The most preferred monoalkenyl aromatic compounds are styrene and a styrene ring substituted with a straight or branched chain alkyl moiety of from 1 to about 12 carbon atoms.

The preferred alkyl esters derived from a saturated alcohol and acrylic or methacrylic acid which may be utilized in the preparation of the polymers for the sustained-release composition of the present invention are acrylate or methacrylate esters derived from an alcohol moiety containing from 1 to about 20, preferably 8 to 20, carbon atoms. Such esters include, for example, butyl methacrylate, butyl acrylate, hexyl acrylate, isobornyl methacrylate, lauryl methacrylate, cetyl methacrylate, eicosyl acrylate, the mixed ester cetyl-eicosyl methacrylate, lauryl methacrylate, stearyl methacrylate, isobornyl acrylate, and lauryl acrylate.

The preferred vinyl esters of aliphatic carboxylic acids used in the preparation of the polymers for the sustained-release composition of the present invention are esters prepared from carboxylic acids containing 2 to about 20, preferably 8 to 20, carbon atoms such as vinyl acetate, vinyl butyrate, vinyl stearate, or vinyl 2-ethylhexoate.

The particularly preferred polymers are crosslinked polymers of styrene and lauryl methacrylate; vinyl toluene and lauryl methacrylate; polymers of tertiary-butylstyrene with lauryl methacrylate, stearyl methacrylate or vinyl stearate; terpolymers of tertiary-butylstyrene, 2-ethylhexyl acrylate and lauryl methacrylate; terpolymers of tertiary-butylstyrene, 2-ethylhexyl acrylate and stearyl methacrylate; polymers of isobornyl methacrylate and lauryl methacrylate; and polymers of vinyl stearate and lauryl methacrylate or isobornyl methacrylate.

More preferably, the sustained-release composition of the present invention comprises a plurality of cross-linked polymer particles wherein the polymer is the polymerization product of two alkyl esters derived from a saturated alcohol and acrylic or methacrylic acid.

Most preferably, the cross-linked polymer particles are cross-linked copolymers of isobornyl methacrylate and lauryl methacrylate. The copolymer is preferably made from 30 to 75, more preferably from 40 to 70, weight percent of isobomylmethacrylate and from 70 to 25, more preferably from 60 to 30 weight percent of laurylmethacrylate, based on the total weight of isobornyl methacrylate and lauryl methacrylate.

The cross-linked polymer particles in general comprise from about 0.01 to about 5 weight percent, preferably about 0.1 to about 2 weight percent, more preferably about 0.3 to about 1 weight percent of cross-linking agent, based on total weight of the polymer. The cross-linking agent can be any di- or poly-functional compound known to be useful as a cross-linking agent such as divinylbenzene, diethylene glycol dimethacrylate, diisopropenylbenzene, diallyl maleate, diallyl phthalate, allyl acrylates, allyl methacrylates, allyl fumarates, allyl itaconates, cyclooctadiene, divinyl phthalates, vinyl isopropenyl benzene, or other di or polyethylenically unsaturated cross-linking agents described, for example, in U.S. Pat. No. 3,520,806.

The particle size diameter of the cross-linked polymer utilized in the sustained-release composition of the present invention may vary, but in general, the particles have a volume average particle size of from about 0.02 to about 1000 micrometers, preferably from about 0.5 to about 500 micrometers, most preferably about 2 to about 100 microns at their smallest diameters. The volume average particle size is measured with a Malvern Mastersizer 2000 light scattering analyzer.

Spherical particles with diameters of from about 2 to about 100 microns are particularly preferred since they are generally invisible to the naked eye and do not scatter light to a high degree. Accordingly such cross-linked polymer particles do not provide an undesirable whitening effect on skin or hair.

The polymer particles are either commercially available or can be produced in a known manner, such as described in U.S. Pat. Nos. 4,489,058 and 4,619,826 and in the International Publications WO 92/00719 and WO 92/00724. Most preferably, the polymers are produced by suspension polymerization.

The cross-linked polymer particles (A) are loaded with the active ingredient (B). The term “loaded with” as used herein is to be understood as having a broad meaning which includes any physical contact of the active ingredient (B) with the plurality of polymer particles (A) which allows a release of the active ingredient from the polymer particles such that the chemical composition of the active ingredient before and after contact with the polymeric particles is substantially unchanged. The “loaded with” as used herein particularly means that the active ingredient is loaded onto or into the polymer particles, e.g., that the active ingredient is adsorbed, absorbed, entrapped and/or imbibed on or in the polymer particles. Typically the active ingredient is imbibed in the polymer particles. The loading is accomplished by adding the active ingredient (B) directly to the plurality of polymer particles (A) in a manner to allow a substantially homogeneous distribution of the active ingredient in the mass of polymer particles, e.g. by spraying, shaking the polymer particles in a liquid ingredient.

The cross-linked polymer particles A can be loaded with a wide variety of one or more active ingredients, for example a skin care compound, such as a moisturizing agent or an emollient, a topical drug, an antioxidant, a dye, a self-tanning compound, an optical brightener, a deodorant, a fragrance, a biocontrol agent, a sunscreen agent or a combination thereof. The active ingredient is preferably a lipophilic compound. More preferably, it has a Hildebrand solubility parameter of from 7 to 12 (cal/cm³)^{1/2}, more preferably from 8.0 to 11.5 (cal/cm³)^{1/2}.

Active ingredients which are particularly useful in personal care compositions, such as skin care or hair care compositions, are known in the art. The term “active” ingredient refers to any ingredient that does not serve as a mere diluent. They can be medical or non-medical ingredients. The term “medical” as used herein means any ingredient that is active in the treatment of the human or animal body by therapy or diagnostic methods. Non-medical ingredients are any ingredients that do not have a therapeutic or diagnostic effect on the human or animal body. Preferred active ingredients are listed below, but their list is not comprehensive. Fragrance includes spices or flavor enhancers which contribute to the overall flavor perception of the composition, such as perfumes or perfume raw materials. The fragrance is typically volatile and has a boiling point of up to 250° C. A disclosure of suitable perfume raw materials, traditionally used in perfumery, can be found in “Perfume and Flavor Chemicals”, Vol. I and II, S. Arctander, Allured Publishing, 1994, ISBN 0-931710-35-5. Examples of fragrance are essential oils and extracts thereof such as mentha, jasmine, camphor, white cedar, bitter orange peel, ryu, turpentine, cinnamon, bergamot, citrus unshiu, calamus, pine, lavender, bay, clove, hiba, eucalyptus, lemon, starflower, thyme, peppermint, rose, sage, sesame, ginger, basil, juniper, lemon grass, rosemary, rosewood, avocado, grape, grapeseed, myrrh, cucumber, watercress, calendula, elder flower, geranium, linden blossom, amaranth, seaweed, ginko, ginseng, carrot, guarana, tea tree, jojoba, comfrey, oatmeal, cocoa, neroli, vanilla, green tea, penny royal, aloe vera, menthol, cineole, eugenol, citral, citronelle, borneol, linalool, geraniol, evening primrose, thymol, spirantol, penene, limonene and terpenoid oils.

Biocontrol agents include, for example, biocides, antimicrobials, bactericides, fungicides, algaecides, mildeweides, disinfecticides, sanitizer-like bleaches, antiseptics, insecticides, insect or moth repellant, vermicides, plant growth hormones and combinations thereof. Typical antimicrobials include glutaraldehyde, cinnamaldehyde and mixtures thereof. Typical insect and moth repellants are perfume ingredients, such as citronellal, citral, N,N-diethyl meta toluamide, Rotundial, 8-acetoxycarvotanacenone, and mixtures thereof.

Preferred emollients or moisturizing agents are glycerin, triglyceride oils, mineral oils, petrolatum, and mixtures thereof, more preferably triglycerides such as sunflower seed oil. Examples of useful emollients are also disclosed in the published patent application WO 03/075879 on page 3, lines 18-31 and page 4, lines 1-11, the teaching of which is incorporated herein by reference.

Examples of useful sunscreen agents are octyl methoxyl cinnamate (Parsol MCX) and butyl methoxy benzoylmethane (Parsol 1789). Other examples of useful sunscreen agents are disclosed in the published patent application WO 03/075879 on page 4, lines 15-31 and on page 5, the teaching of which is incorporated herein by reference.

Other preferred active ingredients are (a) silicone oils and modifications thereof such as linear and cyclic polydimethylsiloxanes; amino, alkyl, alkylaryl, and aryl silicone oils; (b) fats and oils including natural fats and oils, preferably plant fats and oils, such as jojoba, soybean, sunflower, rice bran, avocado, almond, olive, sesame, persic, castor, coconut and mink oils; cacao fat; and animal fats and oils, such as beef tallow and lard; hardened oils obtained by hydrogenating the aforementioned oils; and synthetic mono, di and triglycerides such as myristic acid glyceride and 2-ethylhexanoic acid glyceride; (c) waxes such as carnauba, spermaceti, beeswax, lanolin, and derivatives thereof; (d) plant extracts; (e) hydrocarbons such as liquid paraffins, vaseline, microcrystalline wax, ceresin, squalene, pristan and mineral oil; (f) higher alcohols such as lauryl, cetyl, stearyl, oleyl, behenyl, cholesterol and 2-hexydecanol alcohol; (g) lipids such as cholesterol, ceramides, sucrose esters and pseudo-ceramides as described in European Patent Specification No. 556,957; (h) vitamins, minerals, and skin nutrients such as milk, vitamins A, E, and K; vitamin alkyl esters, including vitamin C alkyl esters; magnesium, calcium, copper, zinc and other metallic components; (i) antiaging compounds, such as alpha hydroxy acids, beta hydroxy acids; or combinations of the listed benefit agents.

Preferably the sustained-release composition of the present invention does not comprise a monomeric release retardant.

The above-described cross-linked polymer particles (A) which are loaded with an active ingredient (B) release the active ingredient (B) over an extended period of time. This means that the release of 50 percent of the total amount of an active ingredient from the sustained-release composition of the present invention comprising the plurality of cross-linked polymer particles (A) takes at least 1.2 times as long, preferably at least 1.3 times as long, more preferably at least 1.5 times as long as the release of 50 percent of the total amount of the same active ingredient from a corresponding composition which does not comprise cross-linked polymer particles (A).

The above-described cross-linked polymer particles (A) which are loaded with an active ingredient (B) are useful in sustained-release compositions, preferably personal care compositions, more preferably a skin care or hair care composition. The preferred skin care or hair care compositions of the present invention are useful for imbibing sebum thus suppressing oily shine on skin and hair while simultaneously delivering an active ingredient to the hair or skin over a time period.

Skin care or hair care compositions, preferably leave-on skin care compositions such as makeup compositions, makeup foundations or lotions; or leave-on hair care composition, such as hair conditioners or foams or rinse-off shampoos, are particularly preferred.

The sustained-release compositions of the present invention preferably comprise the cross-linked polymer particles (A) at an amount of 0.05 to 20 percent, more preferably from 0.1 to 10 percent, based on the total weight of the composition. The given weight relate to the weight of the non-loaded cross-linked polymer particles (A).

The sustained-release compositions of the present invention are generally non-sticky to touch. They are able to absorb or imbibe the sebum responsible for oily shine on the face or the hair of the user and are able to suppress the oily shine over an extended period of time.

The sustained-release composition of the present invention typically comprises a liquid diluent. Preferably water is the main liquid diluent, i.e., water amounts to more than 50 percent, preferably at least 70 percent, more preferably at least 90 percent of the total weight of the liquid diluent. The aqueous composition can also comprise one or more organic diluents such as ethyl alcohol, isopropyl alcohol, higher alcohols or propylene glycol. Depending on the intended use, the sustained-release composition of the present invention may comprise a variety of other components, besides a diluent and the cross-linked polymer particles (A) loaded with the active ingredient (B). Such optional additional components are, for example, active ingredients or adjuvants for which sustained release is not desired, such as cleansing actives, humectants, opacifiers, emollients, emulsifiers, preservatives, actives, thickeners or stabilizers. Such optional additional components preferably are hydrophilic compounds. The type and amount of optional additional components are known in the art and depend on the desired end-use of the sustained-release compositions of the present invention.

The following examples are for illustrative purposes only and are not intended to limit the scope of the present invention. All percentages are by weight unless otherwise specified.

### Example 1

Copolymers of isobornylmethacrylate and laurylmethacrylate which are cross-linked with divinylbenzene are loaded with limonene as described below. The copolymers have the composition listed in Table 1 below and are produced by suspension polymerization as described in U.S. Pat. No. 4,619,826 and U.S. Pat. No. 4,489,058. All weight percentages in Table 1 are based on the total weight of isobornylmethacrylate and laurylmethacrylate.

TABLE 1

| | Weight Percent | Weight Percent | |
|---|---|---|---|
| Polymer | isobornyl | Lauryl | Weight Percent |
| Designation | methacrylate | methacrylate | cross-linking agent |
| 56:44 | 56 | 44 | 0.5 |
| 40:60 | 40 | 60 | 0.5 |

The loading of the cross-linked polymer particles with limonene as the active ingredient is evaluated by placing a 0.15 g sample of the cross-linked polymer particles in small GC (gas chromatography) vials. The beads fill the vials to a height level of 5 mm The vials are then filled with 1.5 g of the active ingredient. Increases in the height level of the beads are recorded at regular time intervals. The cross-linked polymer particles swell when they are loaded with the active ingredient. The swell factor is defined as the ratio of the final equilibrium height to the starting height of 5 mm.

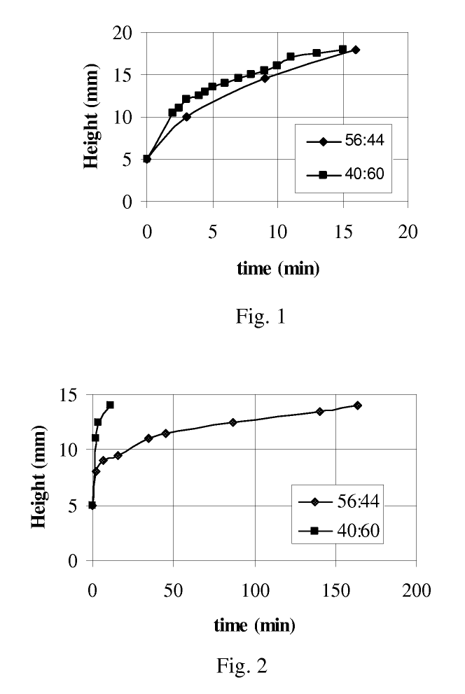

FIG. 1 illustrates the height level of the cross-linked polymer particles listed in Table 1 after they have been contacted with limonene between 0 minutes and more than 20 minutes. Limonene has a solubility parameter of 8.1 (cal/cm³)^{1/2} FIG. 1 illustrates that the cross-linked polymer particles are loaded with limonene. More specifically, limonene is imbibed in the cross-linked polymer particles.

FIG. 2 illustrates the height level of two types of the cross-linked polymer particles listed in Table 1 after they have been contacted with a mixture of olive oil and limonene at a weight ratio of 80:20.

### Examples 2-7

These examples illustrate the sustained release of various fragrances from loaded cross-linked polymer particles as a function of time. In small GC vials, about 0.0025 g of cross-linked copolymer particles are allowed to imbibe about 0.0125 g of fragrance (5 times the weight of cross-linked particles) and the vials are sealed immediately. Based on loading kinetics study, sufficient time is allowed for equilibrium swelling. After such time, about 1.4 g of a mixture of 50 weight percent of isopropanol and 50 weight percent of water is injected with a syringe, the time recorded and vials shaken on a vortex mixer for 1 minute. Immediately after mixing of the ingredients in the vial, the vial is loaded on the auto-sampler of a GC device and 10 micro liters of sample injected into the columns The GC is programmed to withdraw a 10 micro liter sample from the vial every 15 minutes to generate a release profile.

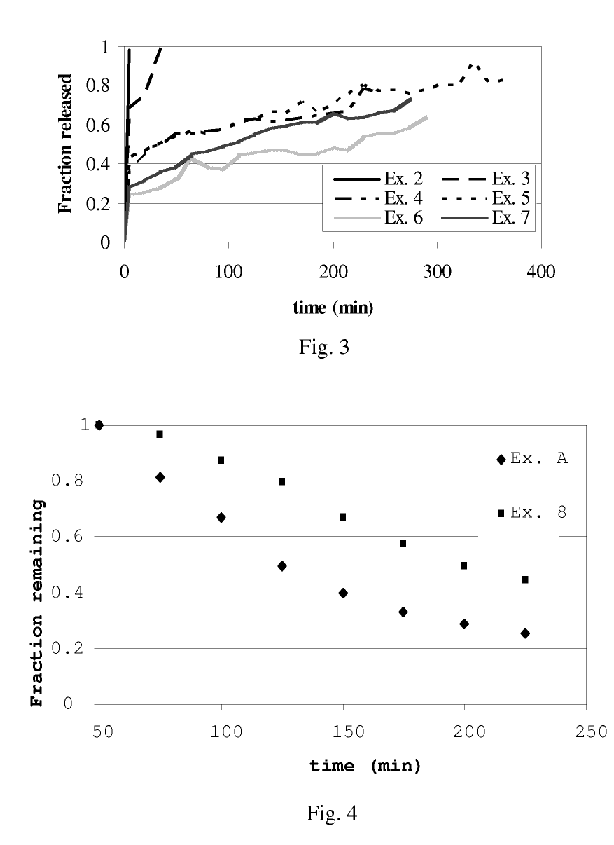

The polymer designations and the fragrances imbibed in the cross-linked polymer particles are listed in Table 2 below. The properties of the cross-linked polymer particles with a given polymer designation are listed in Table 1 above. FIG. 3 illustrates the fraction of released fragrance over time.

TABLE 2

| | Polymer | |
|---|---|---|
| Example | Designation | Fragrance |
| 2 | 40:60 | Limonene |
| 3 | 56:44 | Limonene |
| 4 | 40:60 | 80 percent olive oil + 20 percent limonene |
| 5 | 56:44 | 80 percent olive oil + 20 percent limonene |
| 6 | 40:60 | 80 percent sunflower oil + 20 percent limonene |
| 7 | 56:44 | 80 percent sunflower oil + 20 percent limonene |

### Example 8 and Comparative Example A

Model lotions are produced which have the composition in Table 3 below. Promulgen™ G is an emulsifying agent and stabilizer for hair care and skin care products, commercially available from Noveon, Inc., USA, INCI Name: Stearyl Alcohol (and) Ceteareth 20. Glucam™ P10 is a PPG-10 methyl glucose ether which is commercially available from Noveon. Carbopol™ polymers are crosslinked acrylic acid-based polymers. Methocel E3 LV hypromellose is commercially available from The Dow Chemical Company, has a methoxyl content of 28-30 percent, a hydroxypropoxyl content of 7-12 and a viscosity of 2.4-3.6 cps (m.Pas), measured as a 2 weight percent aqueous solution at 25° C. In Example 8 the cross-linked copolymer particles with the designation 56:44 are loaded with a mixture of Glucam™ P10 and Limonene at a weight ratio of 80:20 before the imbibed particles are added to a mixture of Promulgen™ G and water. In Comparative Example A the mixture of Glucam™ P10 and Limonene at a weight ratio of 80:20 is added without the cross-linked copolymer particles 56:44. The other ingredients are subsequently added.

TABLE 3

| | | Comparative |
|---|---|---|
| | Example 8 | Example A |
| Component | (weight percent) | (weight percent) |
| Water | 86.84 | 85.84 |
| Promulgen ™ G | 4 | 4 |
| a 3% aqueous solution of neu- | 6.66 | 6.66 |
| tralized Carbopol ™ polymer | | |
| Glucam ™ P10:Limonene at a | 2.5 | 2.5 |
| weight ratio of 80:20 | | |
| Cross-linked copolymer 56:44 | 0 | 1 |

Multiple Head Space Extraction (MHE) Gas Chromatography (GC) method is used that simulates drying of VITRO-SKIN to characterize the release profile of limonene. A circular piece of VITRO-SKIN with the lotion applied to it is placed on the bottom of a GC vial with the lotion side facing upwards. It is then sealed. As the fragrance (limonene) evaporates it is collected in the head-space over the skin in the vial. The GC is used to analyze the head-space in the vial to determine the amount of fragrance released. In reality when a human being applies a lotion to the skin the fragrance is released and evaporates into the atmosphere. In the sealed vial to simulate this evaporation from skin, the vial is purged for 10 sec after analysis by the GC. After 20 minutes, the head space is again analyzed for the fragrance content. VITRO-SKIN® is a testing substrate that mimics the surface properties of human skin. It is commercially available from IMS Inc., Orange Conn., USA.

The release profiles of the model lotion of Example 8 and of Comparative Example A are shown in FIG. 4. As illustrated by FIG. 4, the lotion comprising the fragrance imbibed in the cross-linked copolymer 56:44 is released from the lotion in a considerably delayed manner, as compared to the release of the same fragrance from the control formulation wherein the fragrance is not imbibed in the cross-linked copolymer beads.

0.2 g of each of the formulations of Example 8 and Comparative Example A are applied to six samples of 3 cm×6 cm hydrated VITRO-SKIN. Three samples are immediately extracted with ethanol and the ethanol solutions characterized for limonene content. Three samples are allowed to dry for 2 hours and then extracted with ethanol. The fraction remaining after 2 hours is characterized by the ratio of the average extracted limonene after 2 hours to the average extracted limonene immediately after application to the skin for each lotion.

For the lotion of Example 8 this ratio is 0.76. For the lotion of Comparative Example A this ratio is 0.36. This data confirms the substantially delayed release of the active ingredient of the lotion of the present invention, as compared to the release of a comparative lotion wherein the active ingredient is not imbibed in cross-linked polymer particles.

A panel study was conducted by applying the lotion of Example 8 onto the left forearm of two panelists and the lotion of Comparative Example A on the right forearms of the two panelists. Five individuals were asked to smell each forearm and determine the arm from which more fragrance could be perceived. After 30 minutes both lotions were equally perceived, however after 1 hour 70% of the individuals perceived more fragrance on the arm to which the lotion of Example 8 had been applied.

### Examples 9 and 10 and Comparative Example B

To study the release of octyl-methoxycinnamate (OMC) into a solvent, about 0.02 g of the cross-linked polymer particles with the designations 40:60 and 56:44 are imbibed with about 0.1 g of OMC overnight in a jar. The properties of the cross-linked polymer particles with these designations are listed in Table 1 above. OMC is a UV absorber in the 290-320 nm range and therefore can be detected by UV-VIS spectrophotometry. About 40 g of a mixture of 50 weight percent of isopropanol and 50 weight percent of water is added to the jar and mixed on a hand-action shaker for a few minutes. A few drops of this solution is filtered through a syringe filter into a UV-cuvette cell. The absorbance at 290 nm is measured. Based on the calibration curve, the concentration of OMC released is calculated. FIG. 5 shows the release kinetics of OMC from the cross-linked polymer particles. FIG. 5 illustrates the sustained release of the active ingredient from the cross-linked polymer particles. In Comparative Example B, in the absence of cross-linked polymer particles, OMC is immediately released to a mixture of 50 weight percent of isopropanol and 50 weight percent of water.

### Example 11 and Comparative Example C

Model lotions are prepared in the same manner as in Example 8. The compositions of the model lotions are listed in Table 4. In Example 11 OMC is imbibed in the cross-linked copolymer 56:44, whereas the lotion of Comparative Example C does not comprise cross-linked copolymer 56:44.

TABLE 4

| | | Control: Comparative |
|---|---|---|
| | Example 11 | Example C |
| Component | (weight percent) | (weight percent) |
| Water | 83.34 | 84.34 |
| Promulgen ™ G | 4 | 4 |
| a 3% aqueous solution of neu- | 6.66 | 6.66 |
| tralized Carbopol ™ polymer | | |
| OMC | 5 | 5 |
| Cross-linked copolymer 56:44 | 1 | 0 |

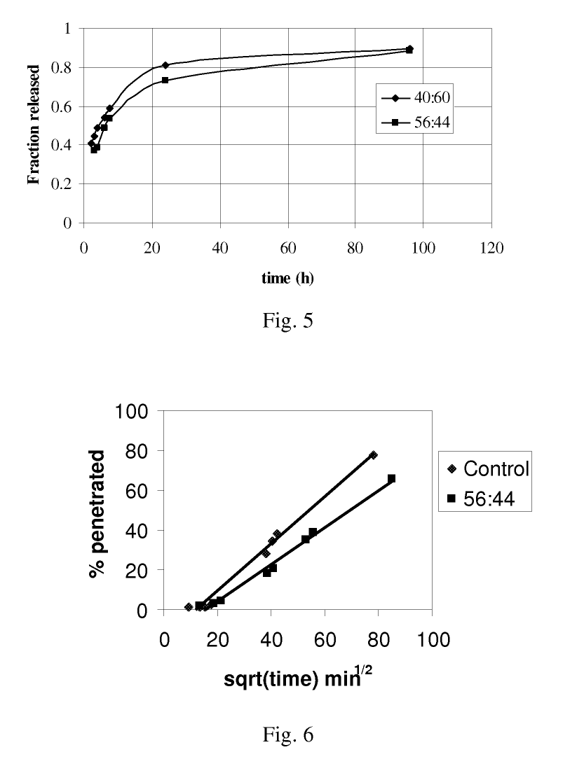

A Franz diffusion cell apparatus, commercially available from PermeGear Inc, Betlehem, Pa., USA is used to study the penetration of OMC from lotions across a synthetic membrane that mimics human skin. A silicone membrane of a thickness of 0.005 inches (0.127 mm) available from Cardiovascular Instruments Corp. in Boston is chosen. 0.3 g of the lotion of Example 11 and 0.3 g of the lotion of Comparative Example C are loaded on different samples of a silicone membrane. The penetration of OMC across the silicone membrane and release of OMC into a mixture of 50 weight percent of isopropanol and 50 weight percent of water is measured. UV spectrometer is used to determine the concentration of OMC in the isopropanol/water mixture at different intervals of time. Three measurements are made for each lotion. The percentage of OMC penetrated across the silicone membrane, the average of three measurements for each lotion, is shown in FIG. 6. Since the percent penetrated OMC can be plotted linearly with square root of time, designated as sqrt(time) in FIG. 6, it implies a pure diffusive release profile. FIG. 6 illustrates that after 24 hours, the lotion of the present invention comprising the active ingredient loaded on the cross-linked copolymer beads is able to delay the release of OMC by nearly 60% as compared to a control lotion of Comparative Example C.

### Example 12 and Comparative Example D

To study the release of Vitamin E, model lotions are prepared in the same manner as in Example 8. The compositions of the model lotions are listed in Table 5. In Example 12 Vitamin E is imbibed in the cross-linked copolymer 56:44, whereas the lotion of Comparative Example D does not comprise cross-linked copolymer 56:44.

TABLE 5

| | | Control: Comparative |
|---|---|---|
| | Example 12 | Example D |
| Component | (weight percent) | (weight percent) |
| Water | 86.34 | 87.34 |
| Promulgen ™ G | 2 | 2 |
| a 3% aqueous solution of neu- | 6.66 | 6.66 |
| tralized Carbopol ™ polymer | | |
| Vitamin E | 4 | 4 |
| Cross-linked copolymer 56:44 | 1 | 0 |

These lotions are loaded on a Franz cell setup and the percentage of Vitamin E penetration across a silicone membrane into isopropanol is measured as described in Example 11. The average results of three Franz cell experiments are shown in FIG. 7. FIG. 7 illustrates that within 24 hours, the amount of Vitamin E released across a silicone membrane from the lotion of the present invention comprising the active ingredient loaded on the cross-linked copolymer beads is only about 15 percent of the amount released from the control lotion of Comparative Example D. These results illustrate that with active ingredients of bulkier molecular size, such as a Vitamin, a sustained release can also be obtained.

### Example 14-16 and Comparative Examples E-G

To study the release of an active ingredient, such as OMC, and the simultaneous sebum control, model lotions are prepared in the same manner as in Example 8. The compositions of the model lotions are listed in Table 6. In the lotions of Examples 14 and 15 OMC is imbibed in the cross-linked copolymer 56:44, whereas the lotions of Comparative Examples E and F do not comprise cross-linked copolymer 56:44.

TABLE 6

| | Ex. 14 | Comp. Ex. E | Ex. 16 | Comp. Ex. F | Ex. 17 | Comp. Ex. G |
|---|---|---|---|---|---|---|
| Component | (wt. %) | (wt. %) | (wt. %) | (wt. %) | (wt. %) | (wt. %) |
| Water | 89.34 | 90.34 | 85.34 | 86.34 | 89.34 | 90.34 |
| Promulgen ™ G | 2 | 2 | 2 | 2 | 2 | 2 |
| a 3% aqueous | 6.66 | 6.66 | 6.66 | 6.66 | 6.66 | 6.66 |
| solution of | | | | | | |
| neutralized | | | | | | |
| Carbopol ™ polymer | | | | | | |
| OMC | 1 | 1 | 5 | 5 | 0 | 0 |
| Glucam ™ P10: | 0 | 0 | 0 | 0 | 1 | 1 |
| Limonene at a | | | | | | |
| weight ratio of | | | | | | |
| 80:20 | | | | | | |
| Cross-linked | 1 | 0 | 1 | 0 | 1 | 0 |
| copolymer 56:44 | | | | | | |

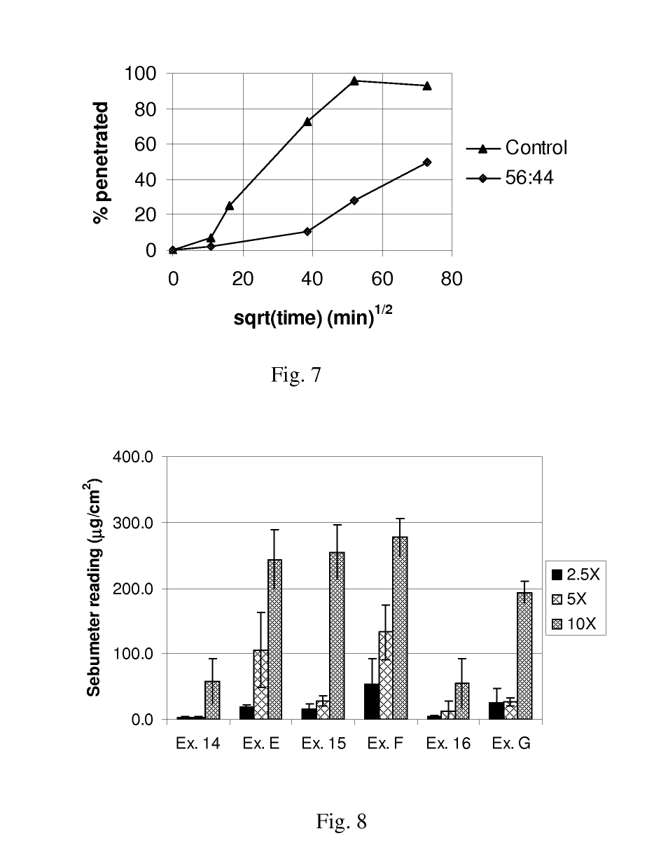

VITRO-SKIN is cut into rectangular pieces of 3 cm×6 cm and hydrated by dipping and swirling in distilled water for a minute. Excess water is removed by pat drying with paper towels. 0.2 g of a lotion is spread uniformly with a gloved finger on the hydrated skin pieces and allowed to dry for an hour. Based on the weight content of cross-linked polymer particles in the lotions of Examples 14-16, the weight of artificial sebum (62% Triolein, 11% Squalene and 27% Oleic acid from Aldrich) that would correspond to 2.5 or 5 or 10 times the weight of the cross-linked polymer particles is applied and spread uniformly on different pieces of skin. After an hour, a sebumeter that works on the principle of grease-spot photometry is used on three spots on each piece of skin to determine an average reading of the oil content in μg/cm². These readings are compared to readings of the control lotions of Comparative Examples E to G with no beads and are shown in FIG. 8. FIG. 8 shows that the sebumeter readings of all lotions with cross-linked copolymer 56:44 are lower than the controls at the same dose level of artificial sebum. FIG. 8 shows that even when the cross-linked polymer particles are loaded with 5 times their weight with an active ingredient, in this case OMC, sebum control is possible. The results in FIG. 8 illustrate the surprising finding that the skin care or hair care compositions of the present invention that are useful for delivering an active ingredient to the hair or skin over an extended period of time is also useful for imbibing and, accordingly, controlling sebum thus suppressing oily shine on skin and hair. Sebum control is even possible if the cross-linked particles are heavily loaded with an active ingredient.

1. A sustained-release composition comprising
(A) a plurality of cross-linked particles of copolymerized isobomylmethacrylate and laurylmethacrylate; and said cross-linked polymer particles being loaded with
(B) a skin care or hair care active ingredient,
the weight ratio of the active ingredient (B) to the polymer particles (A) being from 0.05 to 50:1.

2. The sustained-release composition of claim 1 wherein the weight ratio of the active ingredient (B) to the polymer particles (A) is from 0.2 to 20:1.

3. (canceled)

4. (canceled)

5. The sustained-release composition of any one of claims 1 wherein the cross-linked polymer particles have a volume average particle size of from 2 to 100 microns at their smallest diameters.

6. The sustained-release composition of any one of claims 1 wherein the active ingredient is a lipophilic compound.

7. The sustained-release composition of any one of claims 1 wherein the active ingredient has a solubility parameter of from 7 to 12 (cal/cm³)^{1/2}.

8. The sustained-release composition of any one of claims 1 in the form of a skin care or a hair care composition.

9. A method of releasing an active ingredient to skin or hair over a time period which method comprises contacting the skin or hair with the composition of any one of claims 1.

10. The method of claim 9 wherein an active ingredient is released to skin or hair over a time period and the amount of sebum on skin or hair is controlled.

11. The method of claim 9 wherein the active ingredient is a non-medical ingredient.

12-17. (canceled)
